(19) United States
(12) Patent Application Publication
(10) Pub. No.: US 2007/0238088 A1
(43) Pub. Date: Oct. 11, 2007

(54) Hydrophilic functionalized colloidal silica compositions, methods of making, and uses therefor

(75) Inventors: Slawomir Rubinsztajn, Ballston Spa, NY (US); David Cheney Demoulpied, New Baltimore, NY (US); Omayra Padilla de Jesus, Guilderland, NY (US); Gregory Daryll Goddard, Ballston Spa, NY (US); James Melvin Van Alstine, Stockholm (SE); Jan Anders Larsson, Bromma (SE); Mohan Mark Amaratunga, Clifton Park, NY (US); Nicolas Thevenin, (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 11/392,133

(22) Filed: Mar. 29, 2006

(51) Int. Cl. C12N 5/08; C12Q 1/00; C04B 14/04

(52) U.S. Cl. 435/4; 435/366; 435/325; 106/481; 977/902

(57) ABSTRACT

Disclosed are hydrophilic functionalized silica compositions that are stable and do not show significant pH increases upon heat sterilization. Also provided are methods to make hydrophilic functionalized silica compositions by reacting acidic silica particles with hydrophilic organosilanes. Further provided are methods of separating components in a mixture using hydrophilic functionalized silica compositions.

### BACKGROUND

Disclosed herein are acidic colloidal silica particles modified by hydrophilic organosilanes and compositions comprising multiple acidic colloidal silica particles modified by hydrophilic organosilanes. Also disclosed are methods of making and using functionalized hydrophilic silica particles and compositions.

Aqueous dispersion of colloidal silica particles can be prepared by the polymerization of monosilicic acid from SiO₂ in water. Among other uses, colloidal silica may be used to make gradient density media for separating elements in a mixture, for example separating biological materials by centrifugation.

Colloidal silica has been found to have stability problems. Efforts to increase the stability of the colloidal particles include chemical modification of the surface of silica particles or modification of silica by coating the surface of the particles. Various compositions including dextran, dextran sulfate, polyethylene glycol, polyvinyl alcohol, cellulose, and bovine serum albumen, have been used to coat silica particles to improve stability.

### BRIEF DESCRIPTION

Disclosed herein are stable colloidal silica compositions comprising a plurality of hydrophilic silica particles derived from silica functionalized with a hydrophilic organosilane, wherein the pH of the silica compositions are not increased by heat sterilization. In some embodiments, the pH of the composition is reduced by less than about 2 pH units upon heat sterilization. In other embodiments, the pH of the composition is reduced by less than about 1 pH units upon heat sterilization. In yet other embodiments, the pH of the composition is reduced by less than about 0.5 pH units upon heat sterilization. In other embodiments, the plurality of hydrophilic silica particles is substantially non-agglomerated.

In some embodiments, the plurality of silica particles range in size from about 2 nm to about 250 nm. In some other embodiments, the plurality of silica particles range in size from about 5 nm to about 100 nm. In further embodiments, the plurality of silica particles range in size from about from about 10 nm to about 60 nm.

The hydrophilic organosilane has the general structure I
X—(R)—Si(Y)₃₋ₘR′ₘ Structure I
wherein R is non-hydrolyzable divalent hydrocarbon radical, R′ is a monovalent hydrocarbon radical; m is a whole number equaling 0, 1 or 2; Y is an alkoxy, aryloxy, acyloxy, halogen or amine; and X is an epoxy, an anhydride, an alcohol, a diol, an amine or a sugar. In some embodiments, the organosilane comprises gamma-glycidoxypropyltrimethoxysi lane.

In yet other embodiments, the plurality of silica particles are substantially spherical, substantially elongated, or a combination of substantially spherical and substantially elongated particles.

In further embodiments, the plurality of silica particles produce a linear gradient shape during separation of components having varying buoyant densities. In other embodiments, the plurality of silica particles produces a linear gradient shape during separation of components having varying buoyant densities.

In some embodiments, the total organic content of the colloidal silica composition may be at least 2 weight percent based on total weigh of the composition.

In another aspect, provided herein are methods of making stable colloidal silica compositions. In one embodiment, the method comprises: providing an aqueous dispersion of colloidal silica at a pH range of from about 1 to about 5; providing an organosilane; combining the aqueous dispersion of colloidal silica and the organosilane to form a reaction mixture; and permitting the colloidal silica particles and the organosilane to react. In some embodiments, the pH of the reaction mixture may be adjusted. In further aspects, disclosed herein are colloidal silica particles made by the disclosed methods.

In yet another aspect, disclosed herein is a method of separating components in a mixture comprising: providing a mixture comprising components with varying densities; providing the disclosed colloidal silica composition; combining the mixture with colloidal silica composition; applying a gravitational force to the combination; and optionally, isolating one or more components of the mixture.

These and other features, aspects, and advantages of the present invention will become better understood when the following detailed description is read with reference to the accompanying drawings in which like characters represent like parts throughout the drawings, wherein:

### DESCRIPTION OF THE FIGURES

FIG. 1 shows centrifugal tubes loaded with acidic hydrophilic functionalized colloidal silica from Examples 9, 7, 5, and 10 (shown in panels A, B, C, and D, respectively) in solution following 30 minutes of centrifugation at centrifugal force of 4200 g.

FIG. 2 shows a plot depicting density gradients profiles developed in a fix-45° angle rotor for acidic hydrophilic functionalized colloidal silica from Examples 9, 7, 5, and 10 after 30 minutes of centrifugation at centrifugal force of 4200 g.

FIG. 3 shows density gradients developed in a fix-45′ angle rotor for dispersion of 50 nm acidic hydrophilic functionalized colloidal silica with initial density 1.07 after 5 minutes, 15 minutes, 30 minutes, and 45 minutes centrifugation at centrifugal force of 4200 g.

FIG. 4 shows density gradients developed in a fix-45° angle rotor for 20 nm hydrophilic functionalized colloidal silica with initial density 1.115 after 5 minutes, 15 minutes, 30 minutes, and 45 minutes centrifugation at centrifugal force of 4200 g.

FIG. 5 shows a series of centrifuge tubes containing mixtures of 20 nm and 50 nm hydrophilic functionalized colloidal silica in a dispersion with a starting density of 1.12 g/ml, after 60 minutes of centrifugation at a gravitational force of 2000 g. The tubes in each panel includes mixtures as follows: 100% of 20 nm particles (panel A); 80% of 20 nm and 20% of 50 nm (panel B); 60% of 20 nm and 40% of 50 nm (panel C); 40% of 20 nm and 60% of 50 nm (panel D); 20% of 20 nm and 80% of 50 nm (panel E); 100% of 50 nm (panel F).

### DETAILED DESCRIPTION

Unless otherwise indicated, all numbers expressing quantities of ingredients, properties such as molecular weight, reaction conditions, so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

As used herein, with regard to a component in a mixture, the term “buoyant density” generally refers to the specific gravity of a component disposed in a colloidal dispersion, which effects the equilibrium position of a particular component.

“Colloidal dispersion,” as used herein, refers to a system in which particles of colloidal size (particles which have at least in one direction a dimension roughly between 1 nm and 1 μm) of any nature (i.e., a solid, liquid, or gas) are dispersed in a continuous phase of a different composition (e.g., water, a salt solution, or a sugar solution).

As used herein, the term “coupling agent” refers to a chemical substance capable of interacting with both the surface of the particle such as glass, metal oxide, silica, metals and the continuous phase of the composite materials (water, organic solvents, polymers or resins). Organosilanes are a representative class of coupling agents, which may be described by the formula X—(R)—Si(Y)₃₋ₘR′ₘ where R is non-hydrolyzable divalent hydrocarbon radical, R′ is monovalent hydrocarbon radical, m is a whole number equal 0, 1 or 2, Y is a hydrolysable group such as alkoxy, acyloxy, halogen or amine, X is organic radical that posses functionality which imparts desired characteristic, X could be hydrogen, vinyl, amino, aryloxy, acryloxy, epoxy, anhydride, alcohol, diol. In some embodiments, the coupling agent may be gamma-glycidoxypropyltrimethoxysilane (GLYMO), structure I.

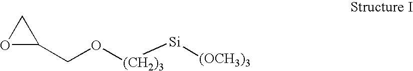

As used herein, the term “density gradient centrifugation” refers to separation techniques based on the density differences in a mixture of components, which takes advantage of the difference in the velocities of different particles as determined by Stoke's law:
vₜ=2R²(pₛ−p)a/(9μ)
where vₜ is the terminal velocity of the particle, R the radius of the particle, a the centrifugal acceleration of the centrifuge, μ the viscosity of the medium, pₛ the density of the particle, and p the density of the medium. The terminal velocity of particle is equal to zero when the density of the particle is equal to the density of the medium. A density gradient may be formed using colloidal dispersions of hydrophilic particles such as colloidal silica, which are used to form a gradient according to their size distribution. The gradients can either be preformed or formed in situ. When the distance of a layer with particular density from the top is plotted against its respective density, the shape of the resulting curve may be represented as a linear gradient, a step gradient, or an “s” shaped gradient. The separation methods disclosed may employ a linear gradient, a step gradient, an “s” shaped gradient depending on the nature of the starting mixture and the desired separation pattern. A component of a mixture will sink or rise until it reaches a position where the density of the surrounding solution is about the same as the density of the particle (the quasi-equilibrium point). A centrifuge may be used to accelerate the process of reaching the quasi-equilibrium point.

As used herein, the term “functional group” refers to an atom or group of atoms, acting as a unit (i.e., a chemical moiety), that has replaced a hydrogen atom in a hydrogen carbon molecule and whose presence imparts characteristic qualities to the resultant molecule. Accordingly, acidic silica may be functionalized by reaction with an organosilane containing hydrophilic functional groups, such as, but not limited to alcohol, diol, or ammonium, and the like, to produce a hydrophilic colloidal silica.

As used herein, the phrase “heat sterilization” of a colloidal dispersion generally refers to heat techniques that may be used to kill microorganisms present in a sample involving elevated temperatures. Heat sterilization techniques may include flowing or pressurized steam produced in a reactor with condenser or an autoclave. A representative heat sterilization scheme may comprise heating a sample in an autoclave to a temperature of about 125° C. for at least about 15 minutes.

The terms “isolated” as used herein generally refer to components of a mixture that have been enriched following processing using the disclosed agents and methods. Although an isolated component may be enriched to purity (i.e., substantially free of other components present in the original mixture), isolated components need not necessarily be so enriched as to be considered pure.

As used herein, the term “nonagglomerated” refers to particles that are substantially loose and separated from each other in whatever state it is present (e.g., dry state or in solution).

“Particle size” as used herein, indicates the average particle size of a range of particle sizes, which may be represented by a size distribution curve. The numerical values for a size distribution curve may be obtained by any art-recognized technique. Thus, particle size may be measured using electron microscopy techniques, such as Transmission Electron Microscopy (TEM), in which particle size may determined by visualizing sample of particles against a calibrated ruler and statistically determining the average size. Particle size may also be determined using optical techniques such as Dynamic Light Scattering (DLS) methods, wherein the scattering of light by particles and the corresponding intensity of scattered light may be used to determine the size distribution. Unless otherwise indicated, intensity average particle sizes distribution disclosed herein are measured using DLS methods and assuming spherical shape of particles that are dispersed in water.

“Physiological pH,” as used herein, refers to the pH value of an aqueous colloid under which physiological moieties, such as, but not limited to, cells including human cells, animal cells, proteins, DNA, RNA, microorganisms such as viruses, bacteria, nucleotides, nucleosides, organelles and other intracellular species remain stable. Physiological is typically characterized as a pH of 7.4. In various embodiments, the pH of the disclosed compositions or dispersions comprising the disclosed compositions may be at physiological pH or near physiological pH so long as the component of interest retains a particular activity. Accordingly, pH values for the disclosed compositions and/or dispersions comprising the disclosed compositions may range from about 5.5 to about 7.5, depending on the particular application.

The term “total organic content,” as used herein, indicates the organic component sample comprising a predetermined mass of particles, which may be measured by subjecting the particle to high temperatures and determining the weight loss from the particle. Unless otherwise indicated, total organic content values disclosed herein are determined using a Thermo-Gravimetric Analyzer (TGA), in which the solid sample of hydrophilic functionalized colloidal silica was prepared by placing 1 g of a water dispersion of hydrophilic functionalized colloidal silicate vacuum oven at 150° C. for 60 min. Thermo-gravimetric analysis (TGA) of the solid sample of hydrophilic functionalized colloidal silica may be carried out using TA Instruments Q5000 TGA in air using following program:

1. Equilibrate at 30° C.

2. Isothermal at 30° C. for 10 min

3. Ramp 10° C./min to 950° C.

As used herein, “total solids content” refer to the amount of solid that is present in a predetermined volume of a colloidal dispersion. Unless otherwise indicated, the values for total solids contents disclosed herein are determined by heating a predetermined weight of a colloidal dispersion in vacuum oven at 150° C. for 60 min and determining the remaining weight, expressed as a percentage.

While only certain features of the invention have been illustrated and described herein, many modifications and changes will occur to those skilled in the art. It is, therefore, to be understood that the appended claims are intended to cover all such modifications and changes as fall within the spirit of the invention.

### SPECIFIC EMBODIMENTS

Disclosed herein are modified silica compositions comprising silica particles functionalized with a hydrophilic organosilane, which are useful in a variety of applications, such as density gradient media. Also disclosed are methods of making the modified silica compositions, and methods of using the disclosed modified silica compositions.

The disclosed modified silica compositions have a sufficient degree of functionality to prevent any gel formation during autoclaving and did not show significant pH increase following heat sterilization and may be autoclaved multiple times without adjusting beyond a pH range from about 5.5 to about 7.5.

Methods of Making Functionalized Hydrophilic Colloidal Silica Compositions

Starting materials for functionalized silica particles may include commercially available various grades of acidic silica particles such as LUDOX® (Aldrich Chemical Co.); SNOWTEX® (Nissan Chemical Co.); and NALCO® from Nalco Co. Several grades of silica exist that may differ based on various parameters, including for example pH of water dispersion, size, or shape. Starting materials used for preparation of hydrophilic colloidal silica of the invention may include commercially available acidic grades of aqueous dispersion of colloidal silica such as, but not limited to, SNOWTEX®-O, SNOWTEX®-040, SNOWTEX®-OS, SNOWTEX®-OL, or NALCO®-1034A.

In general, the disclosed synthesis methods comprise a one-pot reaction in which an acid silica particle is combined with a coupling agent to produce a functionalized hydrophilic colloidal silica composition. In an exemplary embodiment, the reagent includes a hydrophilic organosilane coupling agent having structure I
X—(R)—Si(Y)₃₋ₘR′m Structure I
wherein R is non-hydrolyzable divalent hydrocarbon radical; R′ is monovalent hydrocarbon radical; m is a whole number equal 0, 1 or 2; Y is a hydrolysable group such as alkoxy, acyloxy, halogen or amine; and X is organic radical that posses hydrophilic properties, that includes, but not limited to, an epoxy group, an anhydride group, an alcohol group, a diol group, an amine group, a carbohydrate group, and the like.

In some embodiments, the organosilane comprises an cyclic oxide group having structure II 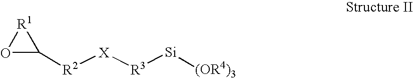
wherein R¹, R², R³ and R⁴ are independently at each occurrence a C₁-C₄₀ aliphatic radical, a C₄-C₄₀ aromatic radical, a C₄-C₄₀ cycloaliphatic radical; and X is selected from the group consisting of N, O, S, or Se. Typical reaction sequence may involve the epoxidization of the corresponding alkene.

Exemplary organosilanes useful as coupling agents include, but are not limited to, (3-glycidoxypropyl)trimethoxysilane, (3-glycidoxypropyl)trichlorosilane, (3-glycidoxypropyl)triethoxysilane, (3-glycidoxypropyl)triacetoxysilane, (2-glycidoxyethyl)trimethoxysilane, (2-glycidoxyethyl)trichlorosilane, (2-glycidoxyethyl)triethoxysilane, (2-glycidoxyethyl)triacetoxysilane, 2-(3,4-epoxycyclohexyl)ethyltrimethoxysilane, 2-(3,4-epoxycyclohexyl)ethyltriethoxysilane bis(2-hydroxyethyl)-3-aminopropyl-trimethoxysilane, bis(2-hydroxyethyl)-3-aminopropyl-triethoxysilane, hydroxymethyltrimethoxysilane, hydroxymethyltriethoxysilane,N-(3-triethoxysilylpropyl)gluconamide and the like.

In some embodiments, the organosilane is selected from the group consisting of organosilanes having structures III-VII:

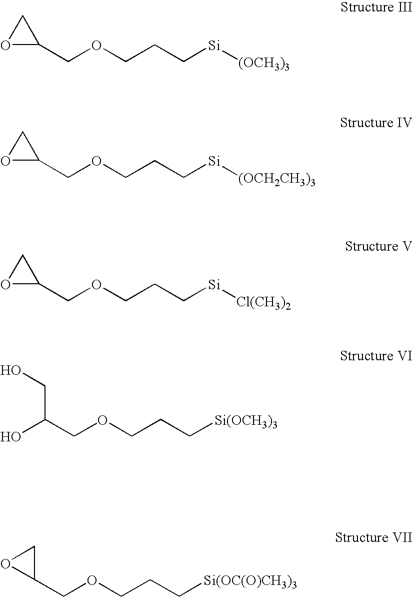

In one embodiment, the organosilane is gamma-glycidoxypropyltrimethoxysilane (GLYMO), structure I, which is commercially available from GE Silicones under the trade name SILQUEST® A-187.

The amount of organosilane to be used in the reaction is dependent on the number of silanol groups available for reaction. One reagent molecule can react and form covalent bonds with one, two, or three surface silanol functional groups.

One of ordinary skill may apply the following equations to determine the amount of the reagent to use to obtain a modified silica particle with high degree of functionalization. Reagent Volume=(Total number of surface silanols/Avogadro's number)/Z)×(reagent molec. wt.) (reagent density) where Z=3 for organotrialkoxysilane. The total number of surface silanol groups within a particular colloidal silica can be calculated as follows: Total surface silanols=volume (ml)×Density (g/ml)×% silica solids (manufacturer's data)×Surface area, nm²/g (manufac. data)×4.5 silanols/nm (value from the Chemistry of Silica: Solubility, Polymerization, Colloid and Surface Properties, and Biochemistry, pp. 633-636, 1979).

The pH of the reaction medium at the beginning, during the reaction and at the end of the reaction is monitored. The pH may be adjusted by the use of buffers, salts, bases, and acids. In one embodiment, the initial pH of the reaction medium at the beginning of the reaction is maintained at a range of from about 1 to about 5. In some other embodiments, the initial pH of the reaction medium is maintained in a range of from about 2 to about 4.

The order and rate of the addition of the reactants are not critical to the practicing the disclosed methods of making hydrophilic functionalized colloidal silica. Thus, the reaction medium may comprise the silica particle to which the organosilane is added or the reaction medium comprises the organosilane to which the silica particle is added. Alternatively, the organosilane and the silica particle may be added to the reaction medium simultaneously. Furthermore, the present methods permit a fast addition of organoalkoxysilane to the stirred water dispersion of acidic colloidal silica, which does not lead to significant agglomeration of the silica particles or the formation of gel.

The time required for the reaction may vary depending on the other reaction conditions such as temperature or atmospheric pressure. Thus, in one embodiment, the time of the reaction may be varied in the range from about 30 minutes to about 48 hours. Similarly, the temperature of the reaction may be varied from about 25° C. to about 100° C. The reaction may also be conducted under super-atmospheric pressures or at atmospheric pressures.

The pH of the reaction mixture, at the end of the reaction, may be optionally adjusted. Thus in some embodiments, the pH may be adjusted to about 7. The pH may be adjusted using a base, such as sodium hydroxide or by use of buffers, such as, but not limited to, phosphate buffered saline (PBS). In embodiments where the modified silica composition is used as a density gradient media for biological applications, a biologically compatible buffer may be employed. Illustrative, but not limiting biologically compatible buffers include, but are not limited to N-2 Hydroxyethylpiperazine-N′-2-Ethanesulfonic acid (HEPES), N—N-bis-2-hydroxyethyl-2-aminoethane sulfonic acid (BES), bis-2-hydroxyethylimino-TRIS-hydroxymethylmethane-2-bis-2-hydroxyethylamino-2-hydroxymethyl-1,3-propanediol (BIS-TRIS), 1,3-bis-[TRIS(hydroxymethyl)methylamino]propane (BIS-TRIS-PROPANE), N2-hydroxyethylpiperazine-N-3-propane sulfonic acid (EPPS), N2-hydroxyethylpiperazine-N2-hydroxypropane sulfonic acid (HEPPSO), 3-N-morpholinopropane sulfonic acid (MOPS), piperazine-N—N-bis-2-ethane sulfonic acid (PIPES), piperazine-N—N-bis-2-hydroxypropane sulfonic acid (POPSO), and 3-N-TRIS-(hydroxymethyl)methylamino-2-hydroxypropane sulfonic acid (TES).

The disclosed colloidal silica compositions may comprise a plurality of silica particles ranging in size from about 2 nm to about 250 nm, about 5 to about 100 nm, or 10 nm to about 60 nm. In further embodiments, the disclosed colloidal silica compositions may comprise a plurality of silica particles combining more than one size population (e.g., a population of about 20 nm particles combined with a population of about 50 nm particles) in varying percentages.

The product of the disclosed methods may be used as such from the reaction medium without employing any isolation procedures. Optionally, the final product may be purified from low molecular weight organic byproducts and residual salt by means known to those skilled in the art such as, but not limited to, dialysis and/or ultrafiltration or the final product may be isolated from the reaction mixture precipitation, drying, and/or centrifugation.

The disclosed methods of making functionalized hydrophilic colloidal silica may be preformed in a single pot process, avoiding time-consuming steps such as slow addition of reaction components and enabling bulk production methods. Furthermore, the disclosed methods reduce gelling and agglomeration of colloidal silica particles in the reaction mixture during the reaction.

The modified silica composition may then be sterilized by using sterilization methods known in the art. In one embodiment, the compositions are sterilized by autoclaving; in another embodiment, the sterilization is achieved by passage of the composition through a 0.2-micron filter. In other embodiments, the sterilization may be achieved by exposing the composition to suitable radiation such as, gamma-radiation, E-beam radiation, or high intensity UV-radiation. The sterilized product can be used under aseptic conditions for sample separation, including cell separation where sterility is required for subsequent processing of the separated component.

Hydrophilic Functionalized Colloidal Silica Compositions

The hydrophilic functionalized colloidal silica made according to the disclosed methods are stable to heat treatment. Thus, in one embodiment, the aqueous dispersion comprising the composition may be subjected to multiple autoclaving steps and the pH of the resulting dispersion does not significantly increase (i.e., remains in the range of physiological pH). Because the pH of the disclosed colloidal silica remain heat treatment does not significantly change, dispersion comprising the disclosed colloidal silica may be heat sterilized and not require addition of an agent to adjust the physiological pH reducing the likelihood of introducing contaminates (e.g., microbes like bacteria, yeast, or fungus) to a dispersion comprising the silica that may damage components of a mixture to be separated.

The hydrophilic functionalized colloidal silica composition may be disposed in various liquid media, such as water, a salt solution (e.g., physiological saline with NaCl), a sugar solution (e.g., sucrose), or a combined salt and sugar solution (e.g., NaCl and sucrose). The salt and/or sugar content may be adapted to enhance viability or structural integrity of one or more components present a sample to be separated. In some embodiments, the liquid media may include a supplement (e.g., a protein or enzyme) that effects one or more component in a predetermined manner (e.g., activating a physiological response).

Due to the presence of hydrophilic groups, functionalized colloidal silica disclosed herein remain substantially nonagglomerated, which enhances the stability of colloidal dispersions comprising the disclosed compositions. Agglomeration or lack thereof, may be demonstrated using Transmission Electron Microscopy (TEM), which may show the individual particles separately or loosely dispersed.

Moreover, the ability of the disclosed colloidal silica compositions to remain unagglomerated following heat sterilization eliminated processing steps (to remove or resuspend agglomerated particles) simplifying preparation of separation compositions and further reducing the likelihood of introducing contaminates during processing steps.

A solution comprising the disclosed hydrophobic colloidal silica compositions may have variable amounts of colloidal silica relative to the volume of the liquid media. The relative mass to volume of a dispersion comprising a hydrophilic colloidal silica disposed in a liquid medium may be characterized by a measurement of the total solids content of the dispersion. In some embodiments, the total solids content may be less than 25 percent by weight based on the total weight of the composition.

Methods of Separating Components in a Mixture

In general, the disclosed methods of separating components in a mixture comprise: (a) providing a mixture of components to be separated; (b) providing the disclosed hydrophilic functionalized silica composition; (c) combining the mixture of components and hydrophilic functionalized silica composition; (d) applying gravitational force to the combination of step (c); and (e) optionally isolating one or more components of the mixture.

The disclosed methods of separating components from a mixture may comprise a single iteration of the steps of: (a) providing a mixture to be separated; (b) providing the disclosed dispersion comprising the hydrophobic functionalized colloidal silica; (c) combining the mixture and dispersion comprising the hydrophobic functionalized colloidal silica; (d) applying gravitational force to the combination of step (c); and (e) optionally isolating one or more components of the mixture. In some embodiments, the steps may be executed in sequence, that is: (a), (b), (c), (d), and then (e). The providing steps need not necessarily occur in (a)-(b) sequences and may, in some embodiments occur as step (b) followed by step (a) prior to the combining step.

In other embodiments, the disclosed methods may comprise multiple iterations of the foregoing steps. Thus, is some alternative embodiments, the disclosed methods may comprise a single iteration of the steps of: (a) providing a mixture to be separated; (b) providing a dispersion comprising the hydrophobic functionalized colloidal silica; (c) combining the mixture and the dispersion; (d) applying gravitational force to form a density gradient, (e) optionally isolating one or more components of the mixture; and (f) repeating each of steps (a)-(e).

The hydrophilic functionalized colloidal silica compositions of the invention may be used, in some embodiments, as density gradient media to separate biological components in a mixture directly derived from an animal or human (e.g., cell types from a blood sample or a plasma sample) or a mixture of cells derived from an in vitro source (e.g., tissue culture). In some specific embodiments, the disclosed methods may be used to separate specific cell types (e.g., blood cells, immune cells, and/or stem cells) from a mixture.

Where the mixture comprises biological components, the separation methods may comprise the steps of: (a) providing a mixture of biological components to be separated; (b) providing the disclosed density gradient media; (c) combining the mixture of biological components and density gradient media; (d) applying gravitational force to the combination of step (c); and (e) optionally isolating one or more biological components of the mixture.

The particle size distribution of the composition may be varied according to the buoyant densities of the components to be separated and the centrifugal force and duration to be used in the separation. The particle size composition of the disclosed functionalized colloidal silica composition may be varied to produce a density gradient optimized to separate components with a range of density equilibrium values. When the mixture of components includes cells that could be damage by excessive gravitational forces, lower gravitational forces (e.g., less than about 2500 g) may be applied in step (d). The user may, in some embodiments, apply higher gravitational forces to a sample comprising whole cells if the viability of the cell following processing is not a concern (e.g., intact or partially intact cells are lysed to release and separate internal cellular components).

Thus, in some application where linear density gradient is required, larger particle sizes are employed to reduce the time and the g-force required to generate a preferred density gradient. In applications, where a step or S-shape density gradient is required, the smaller particle sizes colloidal silica or the mixture of small and large silica particles may be employed to produce a desired density gradient for the separation of biological components.

Separation of components in a sample using the modified silica composition results from the migration of the components to their buoyant densities within the density gradient. The component bands which form at the respective buoyant densities may then be transferred to a new container using any of a variety of techniques, including, for example decanting or aspiration of upper layers. In some embodiments, the component band may be isolated by mechanical extraction using a pipette or withdrawing a band from the side of a tube using a needle and syringe.

An enriched or isolated portion of a sample may include one or more components of a given sample depending upon the relative buoyant density equilibrium values for the components and the specific attributes of the colloidal dispersion (e.g., the size of the silica particles and/or the percent organic content of the dispersion). Furthermore, one or more components in a sample may be sequentially isolated by iteratively repeating the disclosed separation steps under the same or varied conditions to thereby reduce the number of variable components or isolate a single component type. Thus, in some embodiments, the disclosed methods may comprise multiple iterations of steps (a)-(e) or (a)-(f).

The gravitational force applied in the disclosed methods may be applied using a centrifuge, where the sample is placed in a centrifuge tube and spun in a fix-angle or a swing bucket rotor. The gravitational force applied may range from 1 g to 4000 g, preferably from 100 g to 2000 g, the most preferably from 200 g to 800 g.

This invention will be further understood from a consideration of the following Examples. It should be understood, however, that these Examples are given by way of illustration and not by way of limitation and that many changes or alterations may be made in, for example, quantities or choice of material without departing from the scope of this invention as recited in the claims.

### EXAMPLES

Unless otherwise indicated in the following Examples, solids content was determined by a gravimetric method as follows: about 1 g sample of silica dispersion was dispensed on aluminum pan. The pan was placed in vacuum oven at 160° C. for 60 mins to remove any volatiles and determining the remaining weight, expressed as a percentage.

Unless otherwise indicated in the following Examples, particle size and hydrodynamic radius of hydrophilic colloidal silica were determined by Dynamic Light Scatter using Stokes-Einstein/Photon Correlation Spectroscopy. The analysis was performed using a Malvern Instruments HPPS System in water as a solvent.

The density of the silica dispersion was determined to the fourth decimal place by method of U-tube resonant frequency measurement. The measurements were performed using a DMA4500 density meter from Anton Paar.

### Comparative Example 1

### Synthesis of Basic Hydrophilic Functionalized Colloidal Silica According to Prior Art Technique

The process described in U.S. Pat. No. 6,015,483 was adapted here. A round-bottomed 300 ml flask equipped with condenser, magnetic stirrer, thermometer and addition funnel was charged with 90 ml of DI water and 10 ml of glycidoxypropyltrimethoxy silane (Silquest A-187, from GE Silicones). The pH of reaction mixture was adjusted to 2.5 by addition of two drops of 2 N HCl. The reaction mixture was heated at 80° C. for 30 min and subsequently cooled down to 25° C. 100 ml of Ludox HS-40 (40 g SiO2 of basic 12 nm colloidal silica) was added via addition funnel over a period of 15 min to a reaction mixture containing pre-hydrolyzed alkoxysilane. The addition of colloidal silica was followed by the pH adjustment to pH=7 by a drop-wise addition of 8 ml of 0.5 molar HCl. The formation of white fluffy solids has been observed during a neutralization step. The neutral reaction mixture was heated to 80° C. The fluffy white solids dissolved when the batch temperature was about 70° C. After 1 hr of stirring at 80° C. the temperature was raised to 95° C. and the reaction mixture was heated for an additional hour. Subsequently, the reaction mixture was cooled down to RT and filtered through a qualitative paper filter. The pH of the filtrate was adjusted to 9 by dropwise addition of 0.5 N NaOH. The filtered dispersion of hydrophilic functionalized colloidal silica was ultra-filtered through regenerated cellulose Millipore YM100 filter with a cut-off molecular weight of 100 K in a stirred 300 ml Millipore cell with continuous addition of 2 L of DI water. The purified dispersion was sterilized by autoclaving at 125° C. for 30 min.

### Examples 2-5

### Synthesis of Acidic Hydrophilic Functionalized Colloidal Silica According to Prior Art Technique

Round-bottomed 300 ml flask equipped with condenser, magnetic stirrer, thermometer and addition funnel was charged with 90 ml of DI water and 10 ml of glycidoxypropyltrimethoxy silane (Silquest A-187, from GE Silicones). The pH of reaction mixture was adjusted to 2.5 by addition of two drops of 2 N HCl. The reaction mixture was heated at 80° C. for 30 min and subsequently cooled down to 25° C. 100 ml of Snowtex O40 (40 g SiO2 of acidic 20 nm colloidal silica) was added via addition funnel over a period of 15 min to a reaction mixture containing pre-hydrolyzed alkoxysilane. The addition of colloidal silica was followed by the pH adjustment to pH=7 by a drop-wise addition of 0.5 ml of 0.5 molar NaOH. The neutral reaction mixture was heated to 80° C. After 1 hr of stirring at 80° C. the temperature was raised to 95° C. and the reaction mixture was heated for an additional hour. Subsequently, the reaction mixture was cooled down to RT and filtered through a qualitative paper filter. The pH of the filtrate was adjusted to 9 by dropwise addition of 0.5 N NaOH. The filtered dispersion of hydrophilic functionalized colloidal silica was ultra-filtered through regenerated cellulose Millipore YM100 filter with a cut-off molecular weight of 100 K in a stirred 300 ml Millipore cell with continuous addition of 2 L of DI water. The purified dispersion was sterilized by autoclaving at 125° C. for 30 min.

### Examples 6-10

### Synthesis of Acidic Hydrophilic Functionalized Colloidal Silica Using a One-Step Process

A round-bottomed 300 ml flask equipped with condenser, magnetic stirrer, thermometer and addition funnel was charged with 10 g of Snowtex 040 (40 g SiO₂, 12 nm acidic colloidal silica) and 90 ml of DI water. Subsequently, 10 ml of glycidoxypropyltrimethoxy silane (Silquest A-187, from GE Silicones) was added via addition funnel. The pH of the reaction mixture was adjusted to pH=2.5 with 4 drops of 2N HCl. The acidic reaction mixture was heated at 80° C. for 60 min. Subsequently, the solution of colloidal silica was cool down to 60° C. and neutralized by drop-wise addition of 0.6 ml of 0.5 N NaOH. The neutral reaction mixture was heated at 80° C. for one hour and for an additional hour at 95° C. Subsequently, the reaction mixture was cooled down to RT, filtered through a qualitative paper filter and the pH was adjusted to pH=9 by dropwise addition of 0.5 N NaOH. The filtered dispersion of hydrophilic functionalized colloidal silica was ultra-filtered through a regenerated cellulose Millipore YM100 filter with a cut-off molecular weight of 100 K in a stirred 300 ml Millipore cell with continues addition of 2 L of DI water. Finally, the dispersion of hydrophilic functionalized colloidal silica was sterilized by autoclaving at 125° C. for 30 min. Table 1 lists the reactants for experimental runs in Examples 1-10.

TABLE 1

| | | Colloidal silica/ |
|---|---|---|
| Example Number | Run ID | coupling agent |
| Comparative Example 1 | SR83-096 | LUDOX ® HS40/GLYMO |
| Example 2 | SR83-086 | SNOWTEX ® 040/GLYMO |
| Example 3 | SR83-089 | SNOWTEX ® 040/GLYMO |
| Example 4 | SR83-090 | SNOWTEX ® 040/GLYMO |
| Example 5 | SR83-095 | SNOWTEX ® O/GLYMO |
| Example 6 | SR83-091 | SNOWTEX ® O40/GLYMO |
| Example 7 | SR83-092 | NALCO ® 1034A/GLYMO |
| Example 8 | SR83-097 | SNOWTEX ® OS/GLYMO/ |
| | | 70 g water |
| Example 9 | SR83-102 | SNOWTEX ® OS/GLYMO |
| Example 10 | SR83-103 | SNOWTEX ® OL/GLYMO |

TABLE 2

| | | Density | % |
|---|---|---|---|
| Run ID | Filtration/Comments | g/ml | solids |
| SR83-096 | Fast (about 2 min), no gel, filtered ×2 | 1.15 | 25.69 |
| | with #2 filter paper | | |
| SR83-086 | Extremly slow (more than 2 hr), gel | ND | ND |
| | formed on #2 filter paper | | |
| SR83-089 | Very slow (above 30 min), some gel | 1.1716 | 27.1 |
| | formed on #2 filter paper | | |
| SR83-090 | Fast (about 2 min), no gel, filtered ×2 | 1.1463 | 26.02 |
| | with #2 filter paper | | |
| SR83-095 | Slow (above 10 min), no gel, filtered ×2 | 1.15 | 26.25 |
| | with #2 filter paper | | |
| SR83-091 | Fast (about 2 min), no gel, filtered ×2 | 1.1328 | 24.06 |
| | with #2 filter paper | | |
| SR83-092 | Fast (about 2 min), no gel, filtered ×2 | 1.13 | 23.65 |
| | with #2 filter paper | | |
| SR83-097 | Fast (about 2 min), no gel, filtered ×2 | 1.09 | 17.04 |
| | with #2 filter paper | | |
| SR83-102 | Fast (about 5 min), no gel, filtered ×2 | 1.1221 | 22.86 |
| | with #2 filter paper | | |
| SR83-103 | Fast (about 2 min), no gel, filtered ×2 | 1.1262 | 23.75 |
| | with #2 filter paper | | |

### Example 11

### Determination of Ionic Content of Hydrophilic Functionalized Colloidal Silica

The resulting products were characterized for their anionic and cationic content present. Anion content was determined on a Dionex model DX500 ion chromatograph, fitted with an AS17 column, 100 μL injection loop, and a membrane suppressor was used for the analysis of anions. The eluent was potassium hydroxide in 18 MΩ water. A flow rate of 1.5 mL/min. and the elution mode was gradient, from 1 mM to 35 mM KOH. Detection was by suppressed conductivity. The analyte concentrations were calculated by comparison with a series of known standards

Cation content was determined on a Dionex model DX500 ion chromatograph, fitted with a CS12a column, 50 μL injection loop, and a membrane suppressor was used for the analysis of sodium. The eluent was methansulfonic acid (MSA) in 18 MΩ water. A flow rate of 1 mL/min and an elution mode of isocratic at 20 mM MSA were used. Detection was by suppressed conductivity. The analyte concentrations were calculated by comparison with a series of known standards.

The dispersions exposed to lower reaction temperature (experiments SR83-086 and -089, Comparative Examples 2 and 3) were difficult to filter due to presence of micro-gel that plugged the filter paper. The filtered material had a significant level of Na⁺ (about 2500 ppm) and Cl⁻ (about 3500 ppm) as measured by ion chromatography and about 10000 ppm of soluble carbon. Most of these contaminants were removed by ultra-filtration using a regenerated cellulose Millipore YM100 filter with a cut-off molecular weight of 100 KDa in a stirred 300 mL Millipore cell with continued addition of 2 L of deionized water. The filtration step took from 30 to 48 hours using the available laboratory set up. The ultra-filtered dispersions of hydrophilic functionalized colloidal silica particles have low level of Na⁺ (below 30 ppm) and Cl⁻ (below 5 ppm) as measured by ion chromatography. The elemental analysis of filtered water showed only about 300 ppm of the soluble carbon. These results were found to be very similar to the ion content and elemental analysis for the commercial RediGrad®.

### Example 12

### Stability of Hydrophilic Functionalized Colloidal Silica to High Temperature Exposure

The stability of the aqueous suspension of the hydrophilic functionalized silica was determined in the following manner: a 90 g sample of water dispersion of hydrophilic functionalized silica with known density and pH was autoclaved at 125° C. for 30 mins. Subsequently, the weight of the autoclaved sample was adjusted to the initial 90 g by addition of DI water. Then, the sample was filtered and its density as measured above and pH were determined.

All the density separation media that were made described by the process in Examples 1-5 were sterilized under the isotonic conditions to confirm that they had been well functionalized. These hydrophilic functionalized silica particles did not form any solids and their density was almost unchanged, as shown in Table 3, after repetitive autoclaving at 125° C. for 20 min in the presence of 0.15 M NaCl.

TABLE 3

| | | Density of aqeuous suspensions | | | |
|---|---|---|---|---|---|
| | | | After 1 | After 2 | After 3 |
| | | | autoclave | autoclave | autoclave |
| Example | Sample | Initial | run | runs | runs |
| Comparative | SR83-096 | 1.1179 | 1.1135 | 1.1141 | 1.1126 |
| Example 1 | | | | | |
| Example 3 | SR83-089 | 1.111 | 1.1027 | 1.103 | 1.1034 |
| Example 4 | SR83-090 | 1.121 | 1.1175 | 1.1166 | 1.1152 |
| Example 5 | SR83-095 | 1.1195 | 1.1155 | 1.115 | 1.11675 |
| Example 6 | SR83-091 | 1.114 | 1.114 | 1.1143 | 1.11445 |
| Example 7 | SR83-092 | 1.112 | 1.1118 | 1.1104 | 1.11145 |
| Example 8 | SR83-097 | 1.0776 | 1.1147 | 1.1147 | 1.11455 |
| Example 9 | SR83-102 | 1.1092 | 1.105 | 1.1055 | 1.1048 |
| Example 10 | SR83-103 | 1.1035 | 1.1058 | 1.1043 | 1.1035 |

### Example 13

### Determination of pH Stability of Hydrophilic Functionalized Colloidal Silica Particles to Autoclaving at Isotonic Conditions

Acidic hydrophilic functionalized colloidal silica from examples 6, 9, and 10 and colloidal silica from Comparative Example 1 were then tested for their heat stability under isotonic conditions, and was compared with basic hydrophilic functionalized colloidal silica. The stability was determined by measuring the pH of the aqueous medium before and after autoclaving. A 90 g sample of water dispersion of hydrophilic functionalized colloidal silica particles was mixed with 10 g of 1.5 N NaCl or 10 g of 10× Phosphate Buffer Saline (PBS) buffer. The pH of the new solution were measured, following which the sample was autoclaved at 125° C. for 30 mins. A weight of the autoclaved sample was adjusted to the initial 10 g by addition of deionized water. Subsequently, the sample was filtered and its pH were determined.

Table 4 shows the pH of the aqueous dispersions of hydrophilic functionalized colloidal silica particles before and after autoclaving under isotonic conditions.

TABLE 4

| | Input colloidal | | | |
|---|---|---|---|---|
| Examples Numbers | silica | | Initial pH | Final pH |
| Example # 9 | 8 | nm, acidic | 7 | 6.3 |
| Example # 6 | 20 | nm, acidic | 7.24 | 7.1 |
| Example # 10 | 50 | nm, acidic | 7 | 6.5 |
| Comparative Example 1 | 12-20 | nm basic | 7 | 8.27 |

Dispersions of acidic hydrophilic functionalized colloidal silica particles show pH decrease when autoclaved in the presence of 0.15 N NaCl, as compared to the aqueous dispersion of basic hydrophilic functionalized colloidal silica particles that shows an increase in pH. Table 5 shows the pH of the aqueous dispersion of hydrophilic functionalized colloidal silica particles before and after autoclaving in the presence of PBS.

TABLE 5

| Example Numbers | Input colloidal silica | | Initial pH | Final pH |
|---|---|---|---|---|
| Example # 9 | 8 | nm, acidic | 6.96 | 6.82 |
| Example # 6 | 20 | nm, acidic | 7.11 | 6.99 |
| Example # 10 | 50 | nm, acidic | 7.03 | 6.98 |

The Dispersions of acidic hydrophilic functionalized colloidal silica particles having particle sizes 8 nm, 20 nm and 50 nm were found to be quite stable to autoclaving conditions in the presence of PBS as evidenced by a slight pH after the autoclaving.

### Example 14

### Weight Loss of Hydrophilic Functionalized Colloidal Silica Determined by Thermogravimetric Analysis

The organic content of samples was determined by Thermogravimetric Analysis (TGA) using TA Instruments Q5000 TGA in air using following: (1) equilibrate sample at 30° C., (2) hold sample at 30° C. for 10 min, and (3) increase temperature at 10° C./min to 950° C. The solid sample of hydrophilic functionalized colloidal silica particles was prepared by placing 1 g of a water dispersion of hydrophilic functionalized colloidal silica particles at vacuum oven at 150° C. for 60 mins. Table 6 shows the weight loss of hydrophilic functionalized colloidal silica particles.

TABLE 6

| | | |
|---|---|---|
| Example Numbers | Input colloidal silica | % Weight loss |

| | | | |
|---|---|---|---|
| Example # 9 | 8 | nm, acidic | 12.52 |
| Example # 6 | 20 | nm, acidic | 9.19 |
| Example # 10 | 50 | nm, acidic | 2.84 |

The weight loss corresponds to the content of organic coating on the silica particles. The observed weight loss was found to be strongly dependent on particle size.

### Example 15

### Transmission Electron Microscopy (TEM) Imaging of Hydrophilic Functionalized Colloidal Silica

TEM Brightfield transmission electron micrographs were obtained digitally using a FEI CM100 microscope operated at 100 kV employing a side-mounted CCD camera system (AMT). Samples (diluted to less than 1% solids) were transferred to a carbon-coated TEM grid by pipette. Most of the solution was removed by filter paper (acting as a wick), and grids were allowed to dry overnight before examination in the microscope. TEM images of the hydrophilic functionalized colloidal silica particles would give an idea of any possible aggregation effects. The TEM image of the experimental material does not show any aggregation.

### Example 16

### Evaluation of Density Gradient Formation

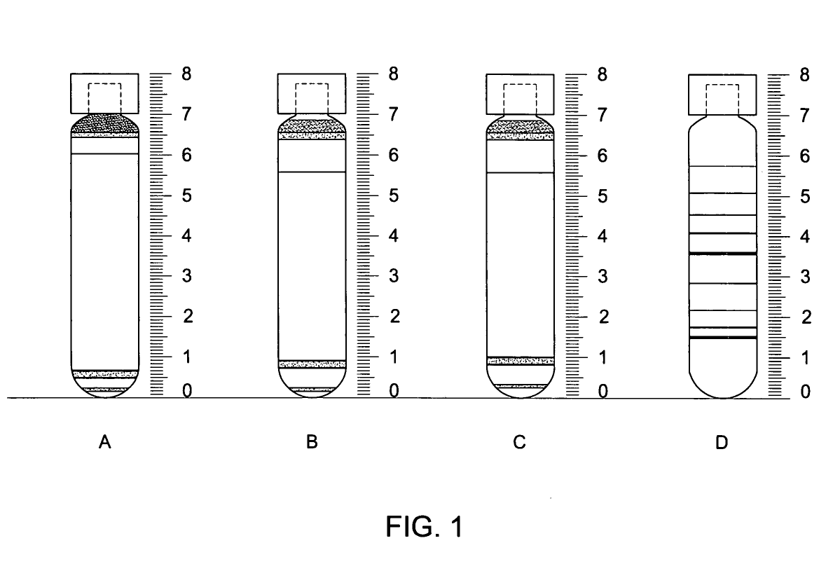

The density gradient formation was evaluated to determine the separation of particles based on the density difference between them by the hydrophilic functionalized colloidal silica particles. The shape of the density gradient formed during centrifugation was determined by means of Colored Density Marker Beads (GE HealthCare). About 150 μl of premixed hydrated beads (vials 1 to 9 of the density marker kit) were added to a 10 mL centrifuge tube containing about 9 mL of density gradient media at a specified initial density. The tubes were then centrifuged in a fixed-angle or swing bucket centrifuge for a desired time at specified g-forces. Most experiments were carried out using a fix 45°-angle centrifuge. FIG. 1 shows centrifugal tubes with acidic hydrophilic functionalized colloidal silica having average particle sizes of 8 nm, 12 nm, 20 nm, and 50 nm following 30 min of centrifugation using centrifugal force of 4200 g. The initial density of all dispersions was 1.12 g/ml. As one could expect, fastest rate of sedimentation was observed for the particles having larger average particle sizes. The variation in the rate of sedimentation was not so strong for hydrophilic functionalized colloidal silica particles having average sizes in the range from 8 nm to 20 nm. All of them had developed similar “S”-type density gradient. Due to the low rate of sedimentation of hydrophilic functionalized colloidal silica particles having average particle sizes of 20 nm, the density gradient undergoes very small changes with time of centrifugation.

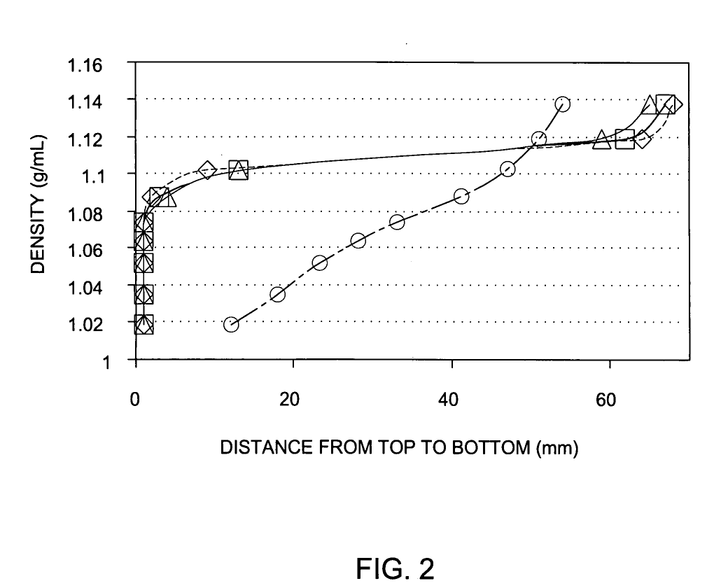

A difference was seen in the sedimentation behavior of the hydrophilic functionalized colloidal silica particles having average particle size of 50 nm (FIGS. 1D and 2). The hydrophilic functionalized colloidal silica particles developed a linear density gradient across the length of the tube after a short centrifugation time and relatively low g force. This behavior may be related to the faster rate of sedimentation of hydrophilic functionalized colloidal silica particles having average particle size of 50 nm.

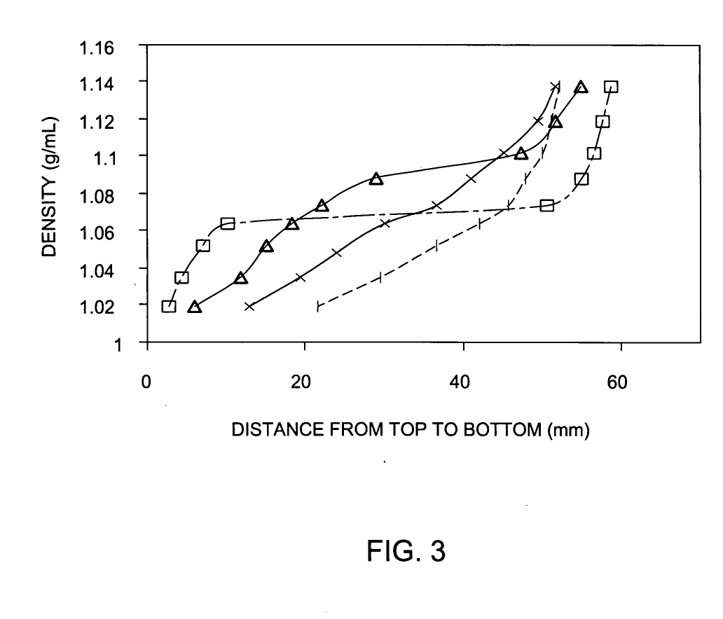

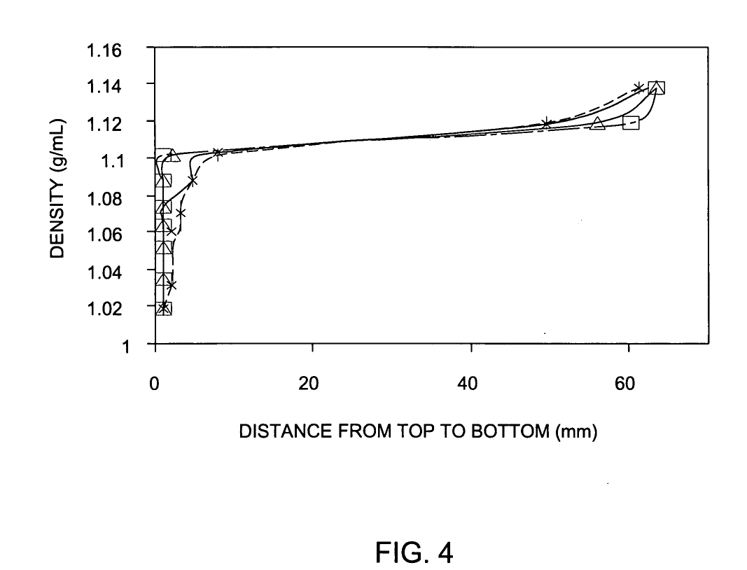

FIGS. 3 and 4 show the effect of centrifuging the samples with the density marker beads as a function of time for the commercial RediGrad™ and acidic hydrophilic functionalized colloidal silica particles having average particle size of 50 nm. FIG. 4 shows that the separation effected by RediGrad™ is almost time independent as evidenced by the plots between the distance from the top versus the density at various times. Even after 45 minutes, the shape of density gradient is almost the same as initial one. FIG. 3 shows separation due to hydrophilic functionalized colloidal silica particles having an average particle size of 50 nm. The developed shape of density gradient is changing much faster, as evidenced by the plots at different times. The initially formed “S”-gradient started to change after less than 15 mins of centrifugation at relatively low g-forces. The centrifugation for 30 mins at these conditions led to the formation of a linear density gradient.

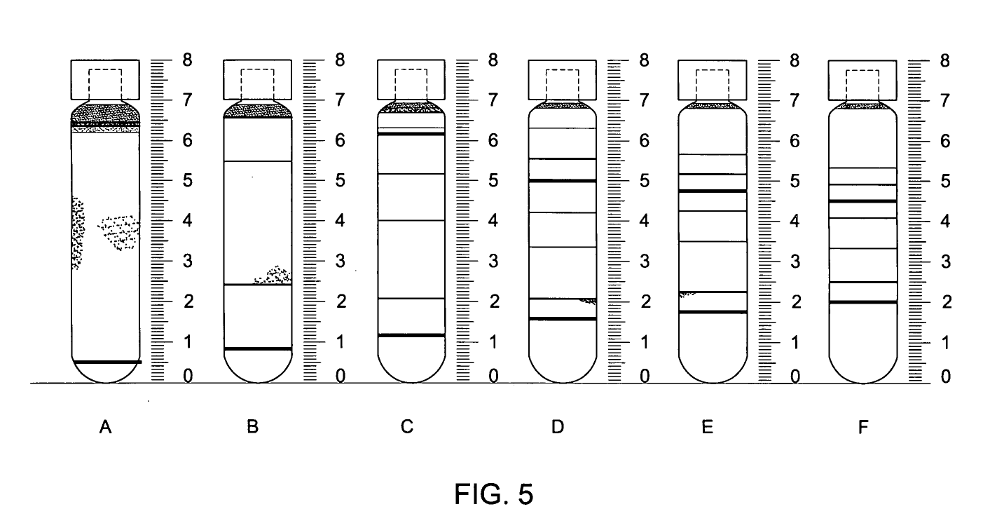

The addition of a small fraction of hydrophilic functionalized colloidal silica particles having an average particle size of 50 nm to the dispersion of RediGrad™ or hydrophilic functionalized colloidal silica particles having average particle size of 20 nm was found to have a strong impact on the formed density gradient (FIG. 5, 2^{nd} and 3^{rd} tube from the left). The formed density gradient maintained the “S”-type character but the density plateau across the length of the tube demonstrates a small slope resulting in formation of a sharp band for the beads with different density.

While only certain features of the invention have been illustrated and described herein, many modifications and changes will occur to those skilled in the art. It is, therefore, to be understood that the appended claims are intended to cover all such modifications and changes as fall within the spirit of the invention.

1. A colloidal silica composition comprising a plurality of hydrophilic silica particles derived from silica functionalized with a hydrophilic organosilane, wherein the pH of the silica composition is not increased by heat sterilization.

2. The colloidal silica of claim 1, comprising wherein the pH is reduced by less than about 2 pH units upon heat sterilization.

3. The colloidal silica of claim 1, comprising wherein the pH is reduced by less than about 1 pH unit upon heat sterilization.

4. The colloidal silica of claim 1, comprising wherein the pH is reduced by less than about 0.5 pH units upon heat sterilization.

5. The colloidal silica composition of claim 1, wherein the plurality of silica particles range in size from about 2 nm to about 250 nm.

6. The colloidal silica composition of claim 1, wherein the plurality of silica particles range in size from about 5 nm to about 100 nm.

7. The colloidal silica composition of claim 1, wherein the plurality of silica particles range in size from about from about 10 nm to about 60 nm.

8. The colloidal silica composition of claim 1, wherein the total organic content of the colloidal silica composition is at least 2 weight percent based on total weight of the composition.

9. The colloidal silica composition of claim 1, wherein the organosilane has structure I
X—(R)—Si(Y)₃₋ₘR′ₘ Structure I
wherein R is non-hydrolyzable divalent hydrocarbon radical, R′ is monovalent hydrocarbon radical, m is a whole number equal 0, 1 or 2, Y is an alkoxy, aryloxy, acyloxy, halogen or amine, X is an epoxy, an anhydride, an alcohol, a diol, an amine or a sugar.

10. The colloidal silica composition of claim 1, wherein the organosilane comprises gamma-glycidoxypropyltrimethoxysi lane.

11. The colloidal silica composition of claim 1, wherein the particle is substantially spherical, substantially elongated, or a combination of substantially spherical particles and substantially elongated particles.

12. The colloidal silica composition of claim 1, wherein the plurality of hydrophilic silica particles are substantially non-agglomerated.

13. The colloidal silica composition of claim 1, wherein the plurality of silica particles produces a linear gradient shape during separation of components having different buoyant densities.

14. The colloidal silica composition of claim 1, wherein the plurality of silica particles produces an “S” shape gradient during separation of components having different buoyant densities.

15. A method of making a colloidal silica composition comprising:
(a) providing a aqueous dispersion of colloidal silica at a pH range of from about 1 to about 5;
(b) providing an organosilane;
(c) combining the aqueous dispersion of colloidal silica and the organosilane to form a reaction mixture;
(d) permitting the colloidal silica particles and the organosilane to react; and
(e) optionally, adjusting the pH of the resulting hydrophilic functionalized colloidal silica particles.

16. The method of claim 15, wherein colloidal silica composition comprises a plurality of silica particles ranging in size from about 2 nm to about 250 nm

17. The method of claim 15, wherein colloidal silica composition comprises a plurality of silica particles ranging in size from about 5 nm to about 100 nm.

18. The method of claim 15, wherein colloidal silica composition comprises a plurality of silica particles ranging in size from about from about 10 nm to about 60 nm.

19. The method of claim 15, wherein the total organic content of the colloidal silica composition is at least 2 weight percent based on total weight of the composition.

20. The method of claim 15, wherein the organosilane has structure I
X—(R)—Si(Y)₃₋ₘR′m Structure I
wherein R is non-hydrolyzable divalent hydrocarbon radical, R′ is monovalent hydrocarbon radical, m is a whole number equal 0, 1 or 2, Y is an alkoxy, acyloxy, aryloxy, halogen or amine, X is an epoxy, an anhydride, an alcohol, a diol, an amine or a sugar.

21. The method of claim 15, wherein the organosilane comprises gamma-glycidoxypropyltrimethoxysilane.

22. The method of claim 15, wherein the colloidal silica composition comprises a plurality of silica particles ranging is substantially spherical, substantially elongated, and a combination of substantially spherical particles and substantially elongated particles.

23. The method of claim 15, wherein the colloidal silica composition comprises a plurality of substantially non-agglomerated silica particles.

24. The colloidal silica composition of claim 15, wherein colloidal silica composition produces a linear gradient shape during separation of components having different densities.

25. The colloidal silica composition of claim 15, wherein the plurality of colloidal silica composition produces an “S” shape gradient during separation of components having different densities.

26. A method of separating components in a mixture comprising:
(a) providing a mixture comprising components with varying densities;
(b) providing the colloidal silica composition of claims 1-15;
(c) combining the mixture and colloidal silica composition;
(d) applying a gravitational force to the combination of step (c); and
(e) optionally, isolating one or more components of the mixture.

27. The method of claim 26, wherein the gravitational force applied ranges from about 1 g to about 4000 g.

28. The method of claim 26, wherein the gravitational force applied ranges from about 100 g to about 2000 g.

29. The method of claim 26, wherein the gravitational force applied ranges from about 200 g to about 800 g.

30. The method of claim 26, wherein the application of gravitational force produces a linear gradient shape.

31. The method of claim 26, wherein the application of gravitational force produces a linear density gradient or S-shape density gradient.

32. The method of claim 26, wherein the mixture to be separated comprises whole cells derived from an animals, whole cells derived from humans, or whole cells derived from a culture.

33. A hydrophilic colloidal silica composition made according to the method of claim 15.
